(19) United States
(12) Patent Application Publication
(10) Pub. No.: US 2024/0360504 A1
(43) Pub. Date: Oct. 31, 2024

(54) CANCER CLASSIFICATION WITH CANCER SIGNAL OF ORIGIN THRESHOLDING

(71) Applicant: Grail, LLC, Menlo Park, CA (US)

(72) Inventors: Anirban NANDI, Lake Forest, IL (US); Qinwen LIU, Fremont, CA (US); Alexander P. FIELDS, Burlingame, CA (US); Jan SCHELLENBERGER, Belmont, CA (US)

(21) Appl. No.: 18/649,559

(22) Filed: Apr. 29, 2024

Related U.S. Application Data
(60) Provisional application No. 63/499,199, filed on Apr. 28, 2023.

(51) Int. Cl. C12Q 1/6874; C12Q 1/6806; C12Q 1/6886; G16B 20/20

(52) U.S. Cl. CPC C12Q 1/6874; C12Q 1/6886; G16B 20/20; C12Q 1/6806

(57) ABSTRACT

Methods and systems for detecting cancer and/or determining a cancer tissue of origin are disclosed. In some embodiments, a multiclass cancer classifier is disclosed that is trained with a plurality of biological samples containing cfDNA fragments. The analytics system derives a feature vector for each sample, and the multiclass classifier predicts a probability likelihood for each of a plurality of cancer signal origin (CSO) classes. In some embodiments, the plurality of CSO classes include hematological subtypes, including both hematological malignancies and precursor conditions. In one embodiment, non-cancer samples having high prediction score are pruned from the training sample set. In another embodiment, the analytics system stratifies samples according to prediction score and applies binary threshold cutoffs determined for each stratum.

### CROSS-REFERENCE TO RELATED APPLICATIONS

This application claims the benefit of priority to U.S. Provisional Patent Application No. 63/499,199, filed Apr. 28, 2023, the contents of which are incorporated herein by reference in their entirety.

### BACKGROUND

Deoxyribonucleic acid (DNA) methylation plays an important role in regulating gene expression. Aberrant DNA methylation has been implicated in many disease processes, including cancer. DNA methylation profiling using methylation sequencing (e.g., whole genome bisulfite sequencing (WGBS)) is increasingly recognized as a valuable diagnostic tool for detection, diagnosis, and/or monitoring of cancer. For example, specific patterns of differentially methylated regions and/or allele specific methylation patterns may be useful as molecular markers for non-invasive diagnostics using circulating cell-free (cf) DNA. However, there remains a need in the art for improved methods for analyzing methylation sequencing data from cell-free DNA for the detection, diagnosis, and/or monitoring of diseases, such as cancer.

### SUMMARY

Early detection of a disease state (such as cancer) in subjects is important as it allows for earlier treatment and therefore a greater chance for survival. Sequencing of DNA fragments in cell-free (cf) DNA sample can be used to identify features that can be used for disease classification. For example, in cancer assessment, cell-free DNA based features (such as presence or absence of somatic variant, methylation status, or other genetic aberrations) from a blood sample can provide insight into whether a subject may have cancer, and further insight on what type of cancer the subject may have. Towards that end, this description includes systems and methods for analyzing cell-free DNA sequencing data for determining a subject's likelihood of having a disease.

An analytics system processes a multitude of sequencing data from a plurality of samples (e.g., a plurality of cancer and non-cancer samples) to identify features that are subsequently utilized for cancer classification. With the sequencing data, the analytics system is able to train and deploy a cancer classifier for generating a cancer prediction for a test sample.

Regarding which training samples are used to train the cancer classifier, the analytics uses training samples that have already been identified and labeled as having one or a number of cancer types, as well as training samples that are from healthy individuals that are labeled as non-cancer. Each training sample includes a set of fragments. For each training sample, the analytics system generates a feature vector, for example, by assigning a score to each of the identified features. The analytics system may group the training samples into sets of one or more training samples for iterative training of the cancer classifier. The analytics system inputs each set of feature vectors into the cancer classifier and adjusts classification parameters in the cancer classifier such that a function of the cancer classifier calculates cancer predictions that accurately predict the labels of the training samples in the set based on the feature vectors and the classification parameters. After iterating the above steps through each set of training samples, the cancer classifier is sufficiently trained.

During deployment, the analytics system generates a feature vector for a test sample in a similar manner to the training samples, e.g., by assigning a score to each of a plurality of features in a feature vector for each of the test samples. Then the analytics system inputs the feature vector for the test sample into the cancer classifier which returns a cancer prediction. In one embodiment, the cancer classifier may be configured as a binary classifier to return a cancer prediction of a likelihood of having or not having cancer. In another embodiment, the cancer classifier may be configured as a multiclass classifier to return a cancer prediction with prediction values for the cancer types being categorized.

The present disclosure, in one embodiment, provides a method for predicting a presence or absence of cancer in a test sample. The method comprises: accessing the test sample having a cancer score and a prediction score for a first tissue label; selecting one of a plurality of strata based on the prediction score, the plurality of strata including a high prediction score stratum and a low prediction score stratum; predicting whether the test sample is associated with a presence or absence of cancer by: transforming the cancer score of the test sample based on a predetermined transformation scale for corresponding prediction score stratum to provide a transformed cancer score; and comparing the transformed cancer score against a predetermined binary threshold cutoff for each stratum. In some embodiments, the predetermined transformation scale for low prediction score stratum is the identity transformation, and the predetermined binary threshold cutoff is identical between the low prediction score stratum and the high prediction score stratum.

In some embodiments, the predetermined binary threshold cutoff is determined by obtaining a holdout set of samples, each sample having the cancer score and the prediction score for the first tissue label; stratifying the holdout set into the high prediction score stratum and the low prediction score stratum based on the prediction scores for the first tissue label of the holdout set of samples; sweeping through a domain of cancer scores at a plurality of candidate binary threshold cutoffs by calculating a true positive rate and a false positive rate for each candidate binary threshold cutoff based on the cancer scores of the samples in the low prediction score stratum, and selecting the binary threshold cutoff from the plurality of candidate binary threshold cutoffs based on a false positive budget for the low prediction score stratum and the calculated false positive rates. sweeping through a domain of cancer scores at a plurality of candidate binary threshold cutoffs by calculating a true positive rate and a false positive rate for each candidate binary threshold cutoff based on the cancer scores of the samples in the high prediction score stratum; selecting the binary threshold cutoff from the plurality of candidate binary threshold cutoffs based on a false positive budget for the high prediction score stratum and the calculated false positive rates, to provide a binary threshold cutoff for the high prediction score stratum; providing one or more candidate transformation scales that transform the binary threshold cutoff for the high prediction score stratum into the predetermined binary threshold cutoff; and selecting the transformation scale based on a false positive budget for the high prediction score stratum. A false positive rate for the predetermined binary threshold cutoff based on the transformed cancer scores of the samples in the high prediction score stratum transformed according to the predetermined transformation scale, may equal the false positive rate for the binary threshold cutoff for the high prediction score stratum based on the cancer scores of the samples in the high prediction score stratum before the transformation.

In some embodiments, the predetermined transformations scale may be a monotonic transformation or in the order of log-odds of the cancer scores. The test sample may comprise a test feature vector determined according to methylation sequencing data of the test sample. The cancer score may be determined by applying a binary cancer classifier to the test feature vector. In some embodiments, the test sample has a prediction score for a second tissue class, wherein selecting one of a plurality of strata is further based on the prediction score for the second tissue label. In some embodiments, the first tissue label is hematological cancer.

In some embodiments, the prediction score is a cancer signal origin (CSO) prediction determined by applying a multiclass cancer classifier to the test feature vector. The CSO prediction may comprise a prediction value for each of a plurality of tissue labels, each prediction value indicating a likelihood that the test sample corresponds to a cancer type associated with the tissue label. In some embodiments, the selecting one of a plurality of strata based on the prediction score for the first tissue label may comprise: determining whether the prediction score for the first tissue label is at or above a prediction value threshold; responsive to determining that the prediction score for the first tissue label is at or above the prediction value threshold, selecting the high prediction score stratum; and responsive to determining that the prediction score for the first tissue label is below the prediction value threshold, selecting the low prediction score stratum. The CSO prediction may indicate one or more top predictions of one or more tissue labels of the plurality of tissue labels, wherein a top prediction of a tissue label indicates that the test sample is predicted to have a cancer type associated with the tissue label of the top prediction. The selecting one of the plurality of strata may comprise: determining whether the first tissue label is a top prediction; responsive to determining that the first tissue label is the top prediction, selecting the high prediction score stratum; and responsive to determining that the first tissue label is not the top prediction, selecting the low prediction score stratum. The selecting one of a plurality of strata may comprise: determining whether the first tissue label is a second top prediction; responsive to determining that the first tissue label is the second top prediction, selecting the high prediction score stratum; and responsive to determining that the first tissue label is not the second top prediction, selecting the low prediction score stratum.

The present disclosure, in one embodiment, provides a method for predicting a presence or absence of cancer in a test sample, the method comprising: accessing the test sample having a cancer score and a prediction score for a first tissue label; selecting one of a plurality of strata based on the prediction score for the first tissue label, the plurality of strata including a first stratum for the first tissue label and a second stratum of for the first tissue label; predicting whether the test sample is associated with a presence or absence of cancer by: i) if the first stratum is selected for the test sample, comparing the cancer score against a predetermined binary threshold cutoff; or ii) if the second stratum is selected for the test sample, transforming the cancer score of the test sample based on a predetermined transformation scale to provide a transformed cancer score; and comparing the transformed cancer score against a predetermined binary threshold cutoff, wherein the predetermined binary threshold cutoff and the predetermined transformation scale is determined based on a holdout set of samples, each sample having a cancer/non-cancer label, the cancer score, and the prediction score for the first tissue label.

In some embodiments, the predetermined binary threshold cutoff is determined by: obtaining the holdout set of samples, each sample having the cancer score and the prediction score for the first tissue label; stratifying the holdout set into the first stratum and the second stratum based on the prediction score for the first tissue label of the holdout set of samples; sweeping through a domain of cancer scores at a plurality of candidate binary threshold cutoffs by calculating a true positive rate and a false positive rate for each candidate binary threshold cutoff based on the cancer scores of the samples in the first stratum, and selecting the binary threshold cutoff from the plurality of candidate binary threshold cutoffs based on a false positive budget for the first stratum and the calculated false positive rates. The predetermined transformation scale may be determined by: sweeping through a domain of cancer scores at a plurality of candidate binary threshold cutoffs by calculating a true positive rate and a false positive rate for each candidate binary threshold cutoff based on the cancer scores of the samples in the second stratum; selecting the binary threshold cutoff from the plurality of candidate binary threshold cutoffs based on a false positive budget for the first stratum and the calculated false positive rates, to provide a binary threshold cutoff for the second stratum; providing one or more candidate transformation scales that transform the binary threshold cutoff for the second stratum into the predetermined binary threshold cutoff; and selecting the transformation scale based on a false positive budget for the second stratum. In some embodiments, a false positive rate for the predetermined binary threshold cutoff based on the transformed cancer scores of the samples in the second stratum transformed according to the predetermined transformations scale, equals the false positive rate for the binary threshold cutoff for the second stratum based on the cancer scores of the samples in the second stratum before the transformation. In some embodiments, the predetermined transformation scale is a monotonic transformation or in the order of log-odds of the cancer scores. In some embodiments, the test sample comprises a test feature vector determined according to methylation sequencing data of the test sample. In some embodiments, the cancer score is determined by applying a binary cancer classifier to the test feature vector.

In some embodiments, the prediction score is a cancer signal origin (CSO) prediction determined by applying a multiclass cancer classifier to the test feature vector. The CSO prediction may comprise a prediction value for each of a plurality of tissue labels, each prediction value indicating a likelihood that the test sample corresponds to a cancer type associated with the tissue label. In some embodiments, selecting one of a plurality of strata based on the prediction score for the first tissue label comprises: determining whether the prediction score for the first tissue label is at or above a prediction value threshold; responsive to determining that the prediction score for the first tissue label is at or above the prediction value threshold, selecting the first stratum; and responsive to determining that the prediction score for the first tissue label is below the prediction value threshold, selecting the second stratum. In some embodiments, the CSO prediction indicates one or more top predictions of one or more tissue labels of the plurality of tissue labels, wherein a top prediction of a tissue label indicates that the test sample is predicted to have a cancer type associated with the tissue label of the top prediction. In some embodiments, selecting one of the plurality of strata comprises: determining whether the first tissue label is a top prediction; responsive to determining that the first tissue label is the top prediction, selecting the first stratum; and responsive to determining that the first tissue label is not the top prediction, selecting the second stratum. In some embodiments, wherein selecting one of a plurality of strata comprises: determining whether the first tissue label is a second top prediction; responsive to determining that the first tissue label is the second top prediction, selecting the first stratum; and responsive to determining that the first tissue label is not the second top prediction, selecting the second stratum. In some embodiments, the test sample has a prediction score for a second tissue class, wherein selecting one of a plurality of strata is further based on the prediction score for the second tissue label.

The present disclosure, in one embodiment, provides a method for predicting a presence or absence of cancer in a test sample, the method comprising: accessing the test sample having a cancer score and a prediction score for a first tissue label; transforming the cancer score of the test sample based on a predetermined transformation scale to provide a transformed cancer score; and predicting whether the test sample is associated with a presence or absence of cancer by comparing the cancer score against a predetermined binary threshold cutoff.

In some embodiments, the predetermined transformation scale is determined by: obtaining a holdout set of non-cancer samples, each sample having the cancer score and the prediction score for the first tissue label; stratifying the holdout set into a high score stratum and a low score stratum based on the prediction scores for the first tissue label of the holdout set of non-cancer samples; sweeping through a domain of cancer scores at a plurality of candidate transformations and a plurality of candidate binary threshold cutoffs by calculating a fraction of false positive samples from the high prediction score stratum to the total false positive samples, wherein the false positive samples have a cancer score higher than each of the binary threshold cutoffs; and selecting the transformation and the binary threshold cutoff from the plurality of candidate binary threshold cutoffs and the plurality of candidate binary threshold cutoffs, based on a target fraction of false positive samples from the high prediction score stratum to the total false positive samples. The predetermined transformation scale may be in the order of log-odds of the cancer scores. The test sample may comprise a test feature vector determined according to methylation sequencing data of the test sample. The cancer score is determined by applying a binary cancer classifier to the test feature vector. In some embodiments, the prediction score is a cancer signal origin (CSO) prediction determined by applying a multiclass cancer classifier to the test feature vector. The CSO prediction may comprise a prediction value for each of a plurality of tissue labels, each prediction value indicating a likelihood that the test sample corresponds to a cancer type associated with the tissue label. In some embodiments, selecting one of a plurality of strata based on the prediction score for the first tissue label comprises: determining whether the prediction score for the first tissue label is at or above a prediction value threshold; responsive to determining that the prediction score for the first tissue label is at or above the prediction value threshold, selecting the high prediction score stratum; and responsive to determining that the prediction score for the first tissue label is below the prediction value threshold, selecting the low prediction score stratum. The CSO prediction may indicate one or more top predictions of one or more tissue labels of the plurality of tissue labels, wherein a top prediction of a tissue label indicates that the test sample is predicted to have a cancer type associated with the tissue label of the top prediction.

In some embodiments, selecting one of the plurality of strata comprises: determining whether the first tissue label is a top prediction; responsive to determining that the first tissue label is the top prediction, selecting the high prediction score stratum; and responsive to determining that the first tissue label is not the top prediction, selecting the low prediction score stratum. In some embodiments, selecting one of a plurality of strata comprises: determining whether the first tissue label is a second top prediction; responsive to determining that the first tissue label is the second top prediction, selecting the high prediction score stratum; and responsive to determining that the first tissue label is not the second top prediction, selecting the low prediction score stratum. In some embodiments, the test sample has a prediction score for a second tissue class, wherein selecting one of a plurality of strata is further based on the prediction score for the second tissue label. In some embodiments, the first tissue label is hematological cancer.

The present disclosure, in one embodiment, provides a system comprising a hardware processor and a non-transitory computer-readable storage medium storing executable instructions that, when executed by the hardware processor, cause the processor to perform steps comprising the method of any of the preceding embodiments.

### BRIEF DESCRIPTION OF THE DRAWINGS

FIG. 1A illustrates a flowchart describing a process of sequencing a fragment of cell-free (cf) DNA to obtain a methylation state vector, according to an embodiment.

FIG. 1B is an illustration of the process of FIG. 1A of sequencing a fragment of cell-free (cf) DNA to obtain a methylation state vector, according to an embodiment.

FIG. 2A illustrates a flowchart of devices for sequencing nucleic acid samples according to one embodiment.

FIG. 2B is a block diagram of an analytics system, according to an embodiment.

FIG. 3A is a flowchart describing a process of training a cancer classifier, according to an embodiment.

FIG. 3B illustrates an example generation of feature vectors used for training the cancer classifier, according to an embodiment.

FIG. 4 illustrates a process for stratifying hematological prediction scores into two strata, in accordance with one or more embodiments.

FIG. 5 illustrates a process of determining binary threshold cutoffs for CSO stratification, in accordance with one or more embodiments.

FIG. 6 illustrates a process for stratifying hematological prediction scores into two strata, in accordance with one or more embodiments.

FIG. 7 illustrates an illustration of the process of calibrating a cancer classifier, according to an embodiment.

FIG. 8 illustrates a flowchart describing a process of predicting cancer presence or cancer absence for a test sample using a binary threshold cutoff determined by CSO stratification, in accordance with one or more embodiments.

FIG. 9 illustrates a process for stratifying hematological prediction scores into two strata, in accordance with one or more embodiments.

FIG. 10 illustrates an illustration of the process of calibrating a cancer classifier, according to an embodiment.

FIG. 11 illustrates a flowchart describing a process of predicting cancer presence or cancer absence for a test sample using a binary threshold cutoff determined by CSO stratification, in accordance with one or more embodiments.

It will be recognized that some or all of the figures are schematic representations for purpose of illustration.

### DETAILED DESCRIPTION

### DNA Methylation and Identification of Cancer

Methylation typically occurs in deoxyribonucleic acid (DNA) when a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Each CpG site may be methylated or unmethylated.

Identification of anomalously methylated fragments, in comparison to healthy individuals, may provide insight into a subject's cancer status. As is well known in the art, DNA methylation anomalies (compared to healthy controls) can cause different effects, which may contribute to cancer. Anomalous DNA methylation can be identified as hypermethylation or hypomethylation, both of which may be indicative of cancer status. Throughout this disclosure, hypermethylation and hypomethylation is characterized for a DNA fragment, if the DNA fragment comprises more than a threshold number of CpG sites with more than a threshold percentage of those CpG sites being methylated or unmethylated. In accordance with the present description, cfDNA fragments from an individual are treated, for example by converting unmethylated cytosines to uracils, sequenced and the sequence reads compared to a reference genome to identify the methylation states at specific CpG sites within the DNA fragments.

Various challenges arise in the identification of anomalously methylated cfDNA fragments. First off, determining a DNA fragment to be anomalously methylated only holds weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site causally influences methylation at a subsequent CpG site. To encapsulate this dependency is another challenge in itself.

Those of skill in the art will appreciate that the principles described herein are equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein are the same, and consequently the inventive concepts described herein are applicable to those other forms of methylation.

### Generating Methylation State Vectors for DNA Fragments

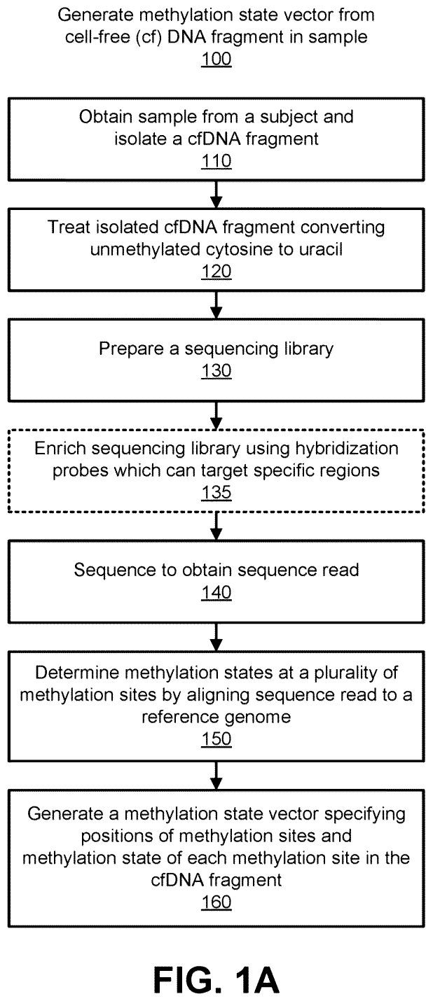

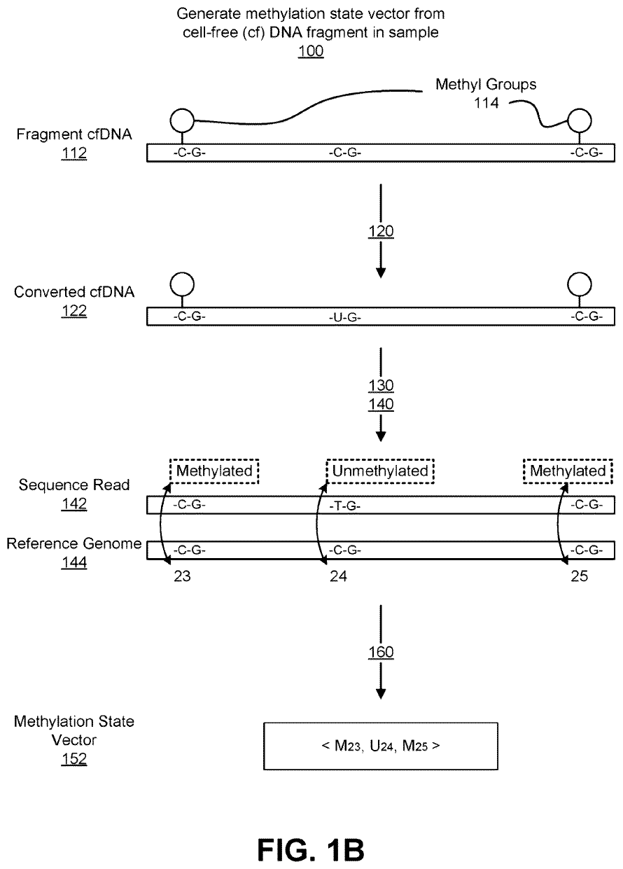

FIG. 1A is a flowchart describing a process 100 of sequencing a fragment of cell-free (cf) DNA to obtain a methylation state vector, according to an embodiment. In step 110, in order to analyze DNA methylation, an analytics system first obtains a sample from an individual comprising a plurality of cfDNA molecules. Generally, samples may be from healthy individuals, subjects known to have or suspected of having cancer, or subjects where no prior information is known. The test sample may be a sample selected from the group consisting of blood, plasma, serum, urine, fecal, and saliva samples. Alternatively, the test sample may comprise a sample selected from the group consisting of whole blood, a blood fraction (e.g., white blood cells (WBCs)), a tissue biopsy, pleural fluid, pericardial fluid, cerebral spinal fluid, and peritoneal fluid. In additional embodiments, the process 100 may be applied to sequence other types of DNA molecules.

From the sample, the analytics system isolates each cfDNA molecule. In step 120, the cfDNA molecules are treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted cfDNA molecules, a sequencing library is prepared in step 130. Optionally, in step 135, the sequencing library may be enriched for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. In one embodiment, the hybridization probes are designed to enrich for DNA molecules that have been treated (e.g., using bisulfite) for conversion of unmethylated cytosines to uracils. Once prepared, the sequencing library or a portion thereof can be sequenced to obtain a plurality of sequence reads, in step 140. The sequence reads may be in a computer-readable, digital format for processing and interpretation by computer software.

From the sequence reads, in step 150, the analytics system determines a location and methylation state for each CpG site based on alignment to a reference genome. In step 160, the analytics system generates a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses.

FIG. 1B is an illustration of the process 100 of FIG. 1A of sequencing a cfDNA molecule to obtain a methylation state vector, according to an embodiment. As an example, the analytics system receives a cfDNA molecule 112 that, in this example, contains three CpG sites. As shown, the first and third CpG sites of the cfDNA molecule 112 are methylated 114. During the treatment step 120, the cfDNA molecule 112 is converted to generate a converted cfDNA molecule 122. During the treatment 120, the second CpG site which was unmethylated has its cytosine converted to uracil. However, the first and third CpG sites were not converted.

After conversion, a sequencing library 130 is prepared and sequenced 140 generating a sequence read 142. The analytics system aligns 150 the sequence read 142 to a reference genome 144. The reference genome 144 provides the context as to what position in a human genome the fragment cfDNA originates from. In this simplified example, the analytics system aligns 150 the sequence read 142 such that the three CpG sites correlate to CpG sites 23, 24, and 25 (arbitrary reference identifiers used for convenience of description). The analytics system thus generates information both on methylation status of all CpG sites on the cfDNA molecule 112 and the position in the human genome that the CpG sites map to. As shown, the CpG sites on sequence read 142 which were methylated are read as cytosines. In this example, the cytosines appear in the sequence read 142 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system generates 160 a methylation state vector 152 for the fragment cfDNA 112. In this example, the resulting methylation state vector 152 is <M₂₃, U₂₄, M₂₅>, wherein M corresponds to a methylated CpG site, U corresponds to an unmethylated CpG site, and the subscript number corresponds to a position of each CpG site in the reference genome.

### Identifying Anomalous Fragments

The analytics system determines anomalous fragments for a sample using the sample's methylation state vectors, comparing it with methylation state vectors from a control group. For each fragment in a sample, the analytics system determines whether the fragment is an anomalous fragment using the methylation state vector corresponding to the fragment. In one embodiment, the analytics system calculates a p-value score for each methylation state vector describing a probability of observing that methylation state vector or other methylation state vectors even less probable in the healthy control group. The analytics system may determine fragments with a methylation state vector having below a threshold p-value score as anomalous fragments. In another embodiment, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

### Analytics System

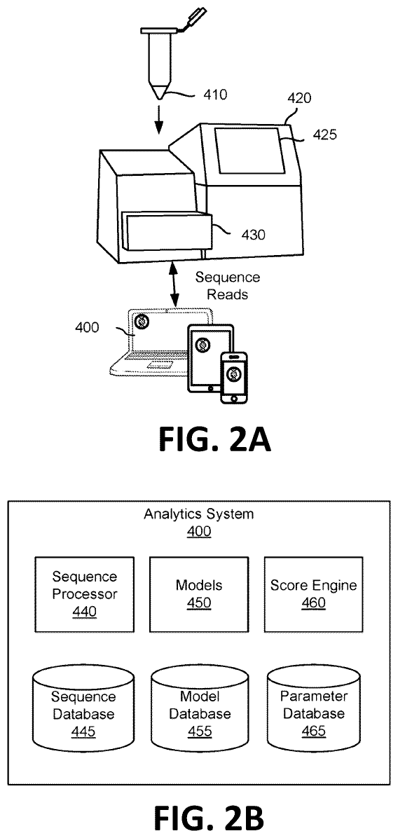

FIG. 2A is a flowchart of devices for sequencing nucleic acid samples according to one embodiment. This illustrative flowchart includes devices such as a sequencer 420 and an analytics system 400. The sequencer 420 and the analytics system 400 may work in tandem to perform one or more steps in the processes described herein.

In various embodiments, the sequencer 420 receives an enriched nucleic acid sample 410. As shown in FIG. 2A, the sequencer 420 can include a graphical user interface 425 that enables user interactions with particular tasks (e.g., initiate sequencing or terminate sequencing) as well as one more loading stations 430 for loading a sequencing cartridge including the enriched fragment samples and/or for loading necessary buffers for performing the sequencing assays. Therefore, once a user of the sequencer 420 has provided the necessary reagents and sequencing cartridge to the loading station 430 of the sequencer 420, the user can initiate sequencing by interacting with the graphical user interface 425 of the sequencer 420. Once initiated, the sequencer 420 performs the sequencing and outputs the sequence reads of the enriched fragments from the nucleic acid sample 410.

In some embodiments, the sequencer 420 is communicatively coupled with the analytics system 400. The analytics system 400 includes some number of computing devices used for processing the sequence reads for various applications such as assessing methylation status at one or more CpG sites, variant calling or quality control. The sequencer 420 may provide the sequence reads in a BAM file format to the analytics system 400. The analytics system 400 can be communicatively coupled to the sequencer 420 through a wireless, wired, or a combination of wireless and wired communication technologies. Generally, the analytics system 400 is configured with a processor and non-transitory computer-readable storage medium storing computer instructions that, when executed by the processor, cause the processor to process the sequence reads or to perform one or more steps of any of the methods or processes disclosed herein.

Referring now to FIG. 2B, FIG. 2B is a block diagram of an analytics system 400 for processing DNA samples according to one embodiment. The analytics system implements one or more computing devices for use in analyzing DNA samples. The analytics system 400 includes a sequence processor 440, sequence database 445, model database 455, models 450, parameter database 465, and score engine 460. In some embodiments, the analytics system 400 performs some or all of the processes 100 of FIG. 1A.

The sequence processor 440 generates methylation state vectors for fragments from a sample. At each CpG site on a fragment, the sequence processor 440 generates a methylation state vector for each fragment specifying a location of the fragment in the reference genome, a number of CpG sites in the fragment, and the methylation state of each CpG site in the fragment whether methylated, unmethylated, or indeterminate via the process 100 of FIG. 1A. The sequence processor 440 may store methylation state vectors for fragments in the sequence database 445. Data in the sequence database 445 may be organized such that the methylation state vectors from a sample are associated to one another.

Further, multiple different models 450 may be stored in the model database 455 or retrieved for use with test samples. In one example, a model is a trained cancer classifier for determining a cancer prediction for a test sample using a feature vector derived from anomalous fragments. The analytics system 400 may train the one or more models 450 and store various trained parameters in the parameter database 465. The analytics system 400 stores the models 450 along with functions in the model database 455.

During inference, the score engine 460 uses the one or more models 450 to return outputs. The score engine 460 accesses the models 450 in the model database 455 along with trained parameters from the parameter database 465. According to each model, the score engine receives an appropriate input for the model and calculates an output based on the received input, the parameters, and a function of each model relating the input and the output. In some use cases, the score engine 460 further calculates metrics correlating to a confidence in the calculated outputs from the model. In other use cases, the score engine 460 calculates other intermediary values for use in the model.

### Training Cancer Classifier

The cancer classifier may be trained to receive a feature vector for a test sample and determine whether the test sample is from a test subject that has cancer or, more specifically, a particular cancer type. The cancer classifier comprises a plurality of classification parameters and a function representing a relation between the feature vector as input and the cancer prediction as output determined by the function operating on the input feature vector with the classification parameters.

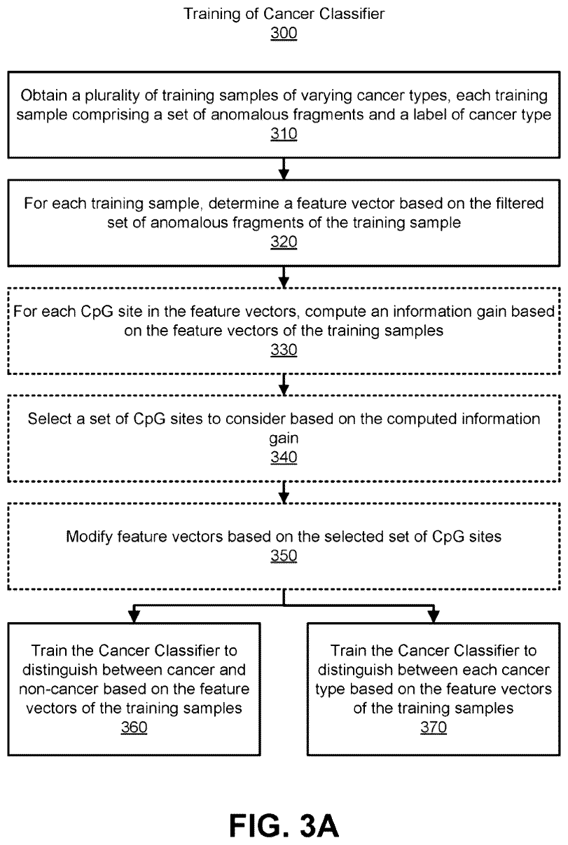

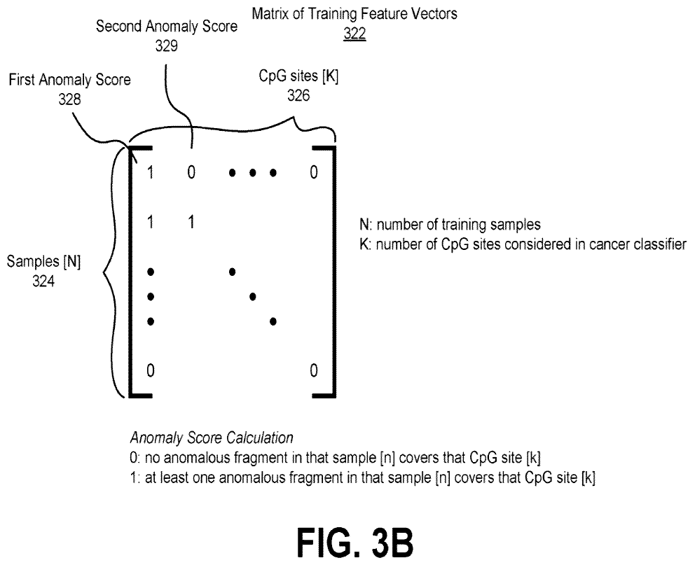

FIG. 3A is a flowchart describing a process 300 of training a cancer classifier, according to an embodiment. In step 310, the analytics system obtains a plurality of training samples each having a set of anomalous fragments and a label of a cancer type. The plurality of training samples includes any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

In step 320, the analytics system determines, for each training sample, a feature vector based on the set of anomalous fragments of the training sample. The analytics system calculates an anomaly score for each CpG site in an initial set of CpG sites. The initial set of CpG sites may be all CpG sites in the human genome or some portion thereof-which may be on the order of 10⁴, 10⁵, 10⁶, 10⁷, 10⁸, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragment in a sample that overlap the CpG site and calculates an anomaly score based on the count of 5.

Once all anomaly scores are determined for a training sample, the analytics system determines the feature vector as a vector of elements including, for each element, one of the anomaly scores associated with one of the CpG sites in an initial set. The analytics system normalizes the anomaly scores of the feature vector based on a coverage of the sample. Here, coverage refers to a median or average sequencing depth over all CpG sites covered by the initial set of CpG sites used in the classifier, or based on the set of anomalous fragments for a given training sample.

As an example, reference is now made to FIG. 3B illustrating a matrix of training feature vectors 322. In this example, the analytics system has identified CpG sites [K] 326 for consideration in generating feature vectors for the cancer classifier. The analytics system selects training samples [N] 324. The analytics system determines a first anomaly score 328 for a first arbitrary CpG site [k1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 328 for the first CpG site as 1, as illustrated in FIG. 3B. Considering a second arbitrary CpG site [k2], the analytics system similarly checks the set of anomalous fragments for at least one that includes the second CpG site [k2]. If the analytics system does not find any such anomalous fragment that includes the second CpG site, the analytics system determines a second anomaly score 329 for the second CpG site [k2] to be 0, as illustrated in FIG. 3B. Once the analytics system determines all the anomaly scores for the initial set of CpG sites, the analytics system determines the feature vector for the first training sample [n1] including the anomaly scores with the feature vector including the first anomaly score 328 of 1 for the first CpG site [k1] and the second anomaly score 329 of 0 for the second CpG site [k2] and subsequent anomaly scores, thus forming a feature vector [1, 0, . . . ].

The analytics system may further limit the CpG sites considered for use in the cancer classifier, because some CpG sites in the initial set of CpG sites may not be as informative as others in distinguishing between cancer types, or may be duplicative with other CpG sites. In one embodiment, in step 330, the analytics system computes an information gain for each cancer type and for each CpG site in the initial set to determine whether to include that CpG site in the classifier. The information gain is computed for training samples with a given cancer type compared to all other samples. That is, how many bits of information about the cancer type are gained if it is known whether there is an anomalous fragment overlapping a particular CpG site. For a given cancer type, the analytics system uses this information to rank CpG sites based on how cancer specific they are, and in step 340, the ranked CpG sites for each cancer type are greedily added (selected) to a selected set of CpG sites based on their rank for use in the cancer classifier. In additional embodiments, the analytics system may consider other selection criteria for selecting informative CpG sites to be used in the cancer classifier. In one embodiment, in step 350, according to the selected set of CpG sites from the initial set, the analytics system may modify the feature vectors of the training samples as needed. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 320 or to the selected set of CpG sites from step 350. In one embodiment, the analytics system trains 360 a binary cancer classifier to distinguish between cancer and non-cancer based on the feature vectors of the training samples. In this manner, the analytics system uses training samples that include both non-cancer samples from healthy individuals and cancer samples from subjects. Each training sample has one of the two labels “cancer” or “non-cancer.” In this embodiment, the classifier outputs a cancer prediction indicating the likelihood of the presence or absence of cancer.

In one embodiment, the analytics system trains 450 a multiclass cancer classifier to distinguish between many cancer types (also referred to as cancer signal origin (CSO) labels). Cancer types include one or more cancers and may include a non-cancer type (may also include any additional other diseases or genetic disorders, etc.). To do so, the analytics system uses the cancer type cohorts and may also include or not include a non-cancer type cohort. In this multi-cancer embodiment, the cancer classifier is trained to determine a cancer prediction (or, more specifically, a CSO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a CSO prediction indicating one or more CSO labels, e.g., a first CSO label with the highest prediction value, a second CSO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier is sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

### Tuning/Calibrating of Cancer Classifier

During use of the cancer classifier, the analytics system may perform operations to calibrate the predictive capabilities of the cancer classifier.

For example, a sample distribution may include one or more non-cancer samples with high prediction score. As used herein, “high prediction score” refers to a sample with a prediction score, e.g., generally for any type of tissue or for a particular cancer type—also referred to as a CSO label, that exceeds some threshold. The prediction score may be determined by a multiclass cancer classifier or other approaches, in comparison to a healthy distribution. Non-cancer samples with high prediction score are outliers in the non-cancer distribution. Some of these high prediction score non-cancer samples may even be pre-stage cancer, early stage cancer, or undiagnosed cancer. As such, non-cancer samples with high-prediction score may muddle the predictive capabilities of the cancer classifier, and the cancer classifier needs to be tuned to “prune” out such outliers.

The analytics system can identify non-cancer samples with high prediction score in at least one CSO label. In one approach of determining high prediction score, a prediction score for a CSO label output by the multiclass cancer classifier is compared against a threshold. Samples with a prediction value above the threshold are deemed to have high prediction score for that CSO label; whereas, samples with a prediction score below the threshold are deemed to not have high prediction score for that CSO label (or low prediction score).

In one embodiment of calibrating the cancer classifier, the sample distribution may be stratified according to prediction score for one or more CSO labels. Then, the analytics system determines a binary threshold cutoff for each resulting stratum with the samples stratified into the stratum. With a test sample, the analytics system places the test sample into a stratum according to the predictions score for one or more CSO labels and predicts the presence or absence of cancer in the test sample with the stratum's binary threshold cutoff.

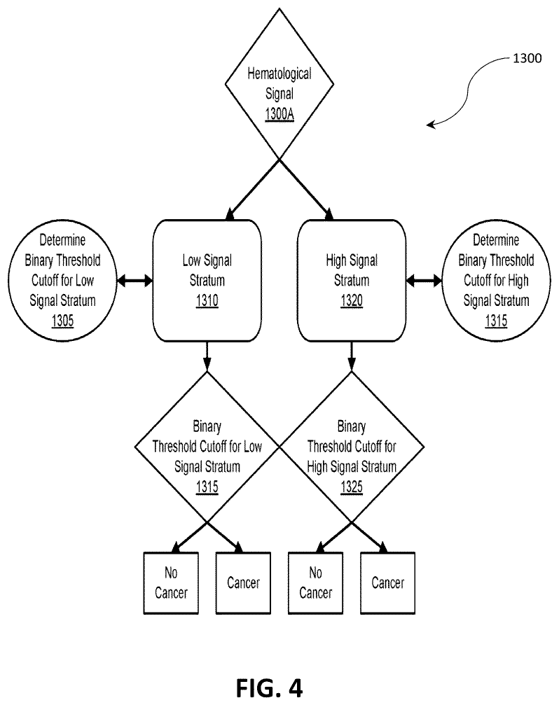

FIG. 4 illustrates a process 1300 for stratifying prediction scores into two strata, in accordance with one or more embodiments. Although the following description describes stratification with a hematological signal/label, the principles may be readily applied to other CSO signals/labels.

In step 1300A, the analytics system stratifies a holdout set of cancer and non-cancer samples according to the prediction scores for hematological cancer into a low prediction score stratum 1310 and a high prediction score stratum 1320. Each sample of the holdout set has a cancer/non-cancer label, a cancer score determined by a binary cancer classifier, and/or a CSO prediction determined by a multiclass cancer classifier. In one embodiment, hematological prediction score for a sample is determined according to a CSO prediction output by a multiclass cancer classifier. In one embodiment, when considering one or more top predictions (e.g., top one, top two, etc.), high hematological prediction score is determined if at least one of the top predictions being considered is one of a hematological subtype (e.g., lymphoid neoplasm subtype and myeloid neoplasm subtype). Other hematological subtypes may be included. As such, if a sample has a CSO prediction with at least one of the top predictions being considered as the lymphoid neoplasm subtype or the myeloid neoplasm subtype, then the sample is determined to have high hematological prediction score. Otherwise, the sample is determined not to have high hematological prediction score. On the other hand, low hematological prediction score is determined if at least one of the top predictions being considered is one of a solid cancer subtype. As such, if a sample has a CSO prediction with at least one of the top predictions being considered as solid cancer subtype(s), then the sample is determined to have low hematological prediction score.

The analytics system determines a binary threshold cutoff for each stratum for predicting presence or absence of cancer of a sample. In step 1305, the samples in the low prediction score stratum 1310 are used by the analytics system to determine a binary threshold cutoff for predicting absence or presence of cancer in samples in the low prediction score stratum 1310. In step 1315, the samples in the high prediction score stratum 1320 are used by the analytics system to determine a binary threshold cutoff for predicting absence or presence of cancer in samples in the high prediction score stratum 1320.

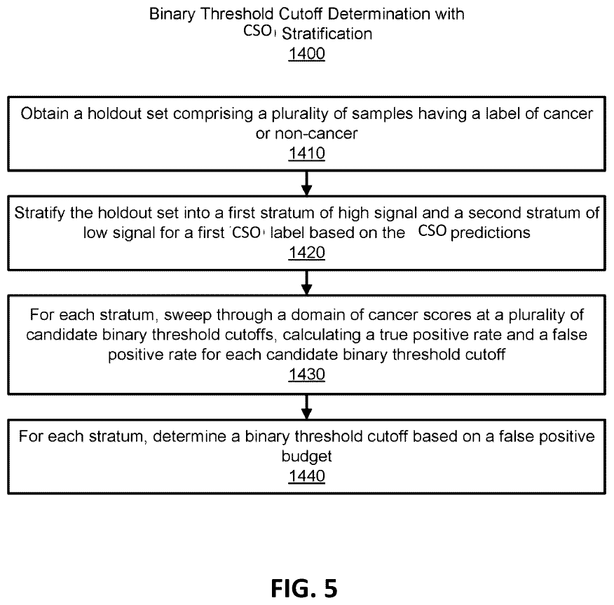

FIG. 5 illustrates a process 1400 for determining binary threshold cutoff for each stratum. In steps 1410 and 1420, the holdout set comprising a plurality of true-non cancer samples may be obtained, and stratified into a first stratum (i.e. high prediction score stratum) or a second stratum (i.e. low prediction score stratum) as described with regard to FIG. 4. In step 1430, with cancer scores for the samples in the high/low prediction score stratum, the analytics system sweeps through a range of candidate binary threshold cutoffs evaluating a false positive rate at each candidate binary threshold cutoff. The candidate binary threshold cutoff with a false positive rate that is closest within the false positive budget for each stratum, is determined to be the binary threshold cutoff for each stratum. The false positive budget for the low prediction score stratum and the false positive budget for the high prediction score stratum may be set according to a ratio of statistical true positive rates of the strata. The ratio aims to suppress the false positive rate in the high prediction score stratum.

In most instances, the binary threshold cutoff for the high prediction score stratum and the binary threshold cutoff for the low prediction score stratum may be different. For example, the binary threshold cutoff for the high prediction score stratum may be higher than the binary threshold cutoff for the low prediction score stratum. Such difference between the thresholds is attributed to the confounding hematological prediction score. That is, there are many non-cancer cells (e.g. lymphoid and myeloid cells) that exhibit cancer-like, high hematological cancer prediction score, having a high cancer score, and thus the threshold cutoff may be higher for the high prediction score stratum.

In some instances, two different thresholds for each of the strata may be maintained and used separately for test samples to predict cancer presence depending on whether the test samples belong to the high prediction score stratum or the low prediction score stratum. For example, as shown in FIG. 4, the binary threshold cutoff for low prediction score stratum may be applied to the samples that are assigned to the low prediction score stratum, and the binary threshold cutoff for high prediction score stratum may be applied to the samples that are assigned to the high prediction score stratum, to determine presence of cancer or not. However, using two different thresholds may be confusing and reduces the interpretability of the results. It may be more difficult to compare the performance of cancer classifier between the high prediction score stratum and the low prediction score stratum. Therefore, there is a benefit to have a single threshold that can be universally used for all samples, regardless of whether it belongs to the high prediction score stratum (e.g. hematological CSO), or the low prediction score stratum (e.g. solid CSO).

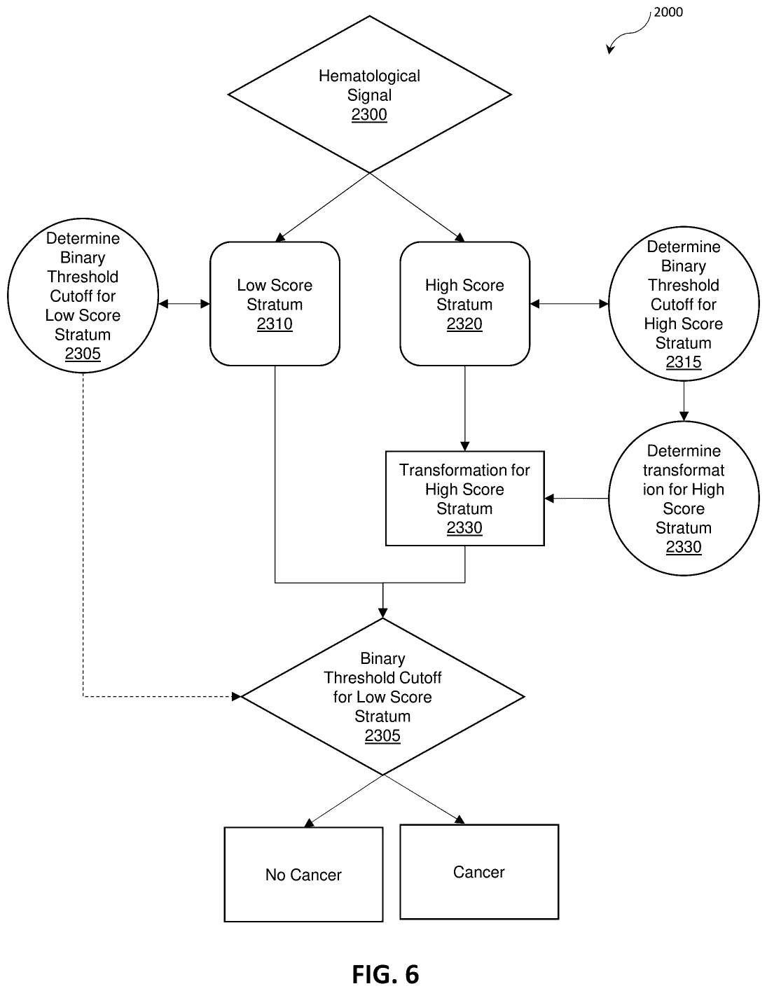

To homogenize two different thresholds, the cancer classifier may be trained to calibrate by shifting cancer scores of samples that belong to the high prediction score stratum and/or the low prediction score stratum. FIG. 6 illustrates a process 2000 for stratifying prediction scores into two strata, in accordance with one or more embodiments where such shifting is made. In step 2300, the analytics system stratifies a holdout set of non-cancer samples according to the hematological prediction score into a low prediction score stratum 2310 and a high prediction score stratum 2320. Each sample of the holdout set has a non-cancer label, a cancer score determined by a binary cancer classifier, and/or a CSO prediction determined by a multiclass cancer classifier. In one embodiment, hematological prediction score for a sample is determined according to a CSO prediction output by a multiclass cancer classifier. In one embodiment, when considering one or more top predictions (e.g., top one, top two, etc.), high hematological prediction score is determined if at least one of the top predictions being considered is one of a hematological subtype (e.g., lymphoid neoplasm subtype and myeloid neoplasm subtype). Other hematological subtypes may be included. As such, if a sample has a CSO prediction with at least one of the top predictions being considered as the lymphoid neoplasm subtype or the myeloid neoplasm subtype, then the sample is determined to have high hematological prediction score. Otherwise, the sample is determined not to have high hematological prediction score.

In step 2330, cancer scores of samples of the high prediction score stratum can be transformed, according to certain rules, formula, or scale, such that the binary threshold cutoff for the high prediction score stratum (adjusted as a result of the transformation as well) is equalized with the binary threshold cutoff for the low prediction score stratum. In an embodiment, the cancer scores of samples of the low prediction score stratum may be transformed.

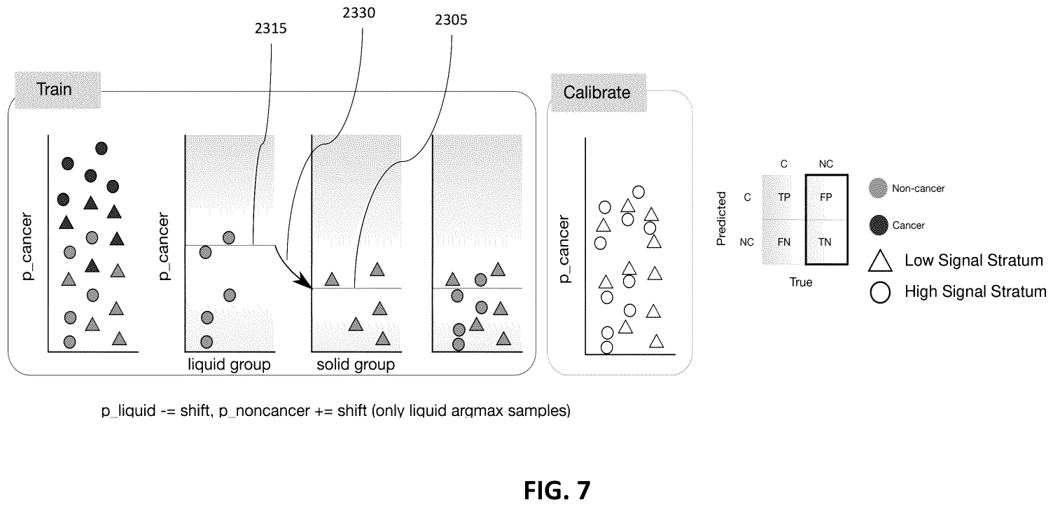

While the transformation shifts the cancer score of samples and also the binary threshold cutoff, to maintain the target specificity and false positive rate, the transformation may not alter the true positive rate and the false positive rate within each stratum. In other words, the relative level or position of the threshold cutoff within the sample distribution needs to stay same before and after the transformation. FIG. 7 schematically illustrates the transformation 2330 according to one embodiment. In FIG. 7, there are 5 true non-cancer samples for the high prediction score stratum (circle) and 5 true non-cancer samples for the low prediction score stratum (triangle). Before the transformation 2330, the high prediction score stratum shows 20% false positive rate (one non-cancer circle above the threshold cutoff 2315). After the transformation 2330, the high prediction score stratum still shows 20% false positive rate relative to the threshold cutoff for low prediction score stratum 2305, as relative “order” of the cancer scores of the samples and the threshold cutoff has not been changed. To achieve this, the transformation 2330 may be a monotonic transformation, such that the relative “order” or ranking of the cancer scores of the samples in the high prediction score stratum 2320 may not be changed.

The scale or formula for the transformation 2330 may be determined by any suitable methods, as far as the false positive rate for the samples in the transformed stratum (i.e. high prediction score stratum) is maintained. In some embodiments, the analytics system may sweep through stored candidate formulas and candidate scales to find a transformation that satisfies this condition.

In some embodiments, the analytics system may determine the scale or formula of the transformation 2330 based on one or more of following factors: cancer scores distribution of holdout set samples in the high prediction score stratum; cancer scores distribution of samples in the low prediction score stratum; the binary threshold cutoff for the high prediction score stratum; and the binary threshold cutoff for the low prediction score stratum. For example, the analytic system may determine the scale based on the difference between the binary threshold cutoffs for the two strata.

In some embodiments, the difference of log odds of the binary threshold cutoffs for the two strata is be calculated, and cancer scores of samples of the high prediction score stratum are transformed in log odds scale according to the difference. In some embodiments, under the transformation 2330, log odds of each cancer score for a stratum may be shifted upwards or downwards by certain constant amount. In some embodiments, under the transformation 2330, each cancer score for a stratum (e.g. high prediction score stratum) may be shifted by certain amount.

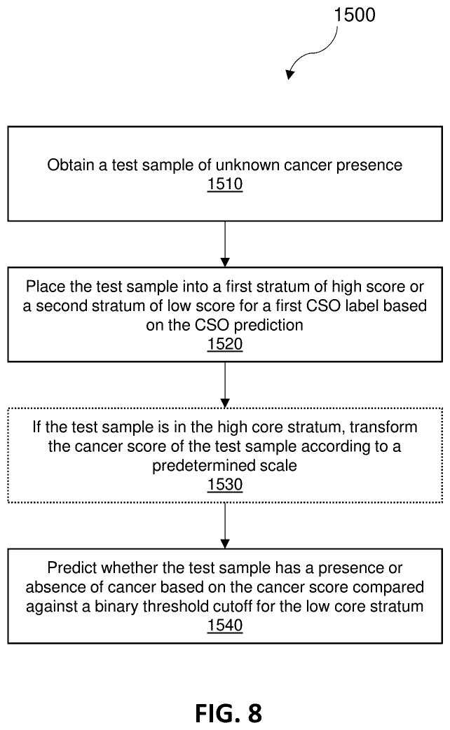

Once the analytics system determines the scale and/or formula for the transformation, and the binary threshold cutoffs, the cancer classifier may test a sample. FIG. 8 illustrates a process 1500 for the binary cancer prediction of the test sample. In step 1510, a test sample of unknown cancer presence may be obtained. The test sample may be processed to provide the CSO signals, including the hematological prediction score. Then, in step 1520, the analytics system places the test sample into either the low prediction score stratum or the high prediction score stratum according to its hematological cancer prediction score. If the test sample is placed in the low prediction score stratum, then in step 1540, the analytics system applies the binary threshold cutoff for the low prediction score stratum 2305 to the cancer score of the test sample. If the cancer score is greater than or equal to the binary threshold cutoff for the low prediction score stratum, then the analytics system returns a prediction of cancer presence in the test sample, and returns a prediction of no cancer otherwise.

In step 1530, if the test sample is placed in the high prediction score stratum 2320, then the cancer score of the test sample is transformed according to the transformation 2330 to provide a transformed cancer score. Then, in step 1540, the analytics system applies the binary threshold cutoff for the low prediction score stratum to the transformed cancer score of the test sample. It the transformed cancer score is greater than or equal to the binary threshold cutoff for the low prediction score stratum 2310, then the analytics system returns a prediction of cancer presence in the test sample, and returns a prediction of no cancer otherwise.

Even though the transformation is described with regard to the embodiment illustrated in FIGS. 4 and 8, where the transformation is applied only to the high prediction score stratum, it would be understood by a person skilled in the art that such transformation may not be limited to the high prediction score stratum, and it may be applied to either or both of the high/low prediction score stratum. In some embodiments, the transformation of the cancer score of the samples may be applied to samples in the high and/or low prediction score strata to enable that a single binary threshold cutoff can be applied to samples distributed to any of the strata to determine presence of cancer or not. Further, the such transformation may be applied to samples in one or more strata, in an embodiment where there are more than two prediction score stratum (e.g., low/middle/high prediction score stratum), to enable that a single binary threshold cutoff can be applied to samples distributed to any of the strata to determine presence of cancer or not.

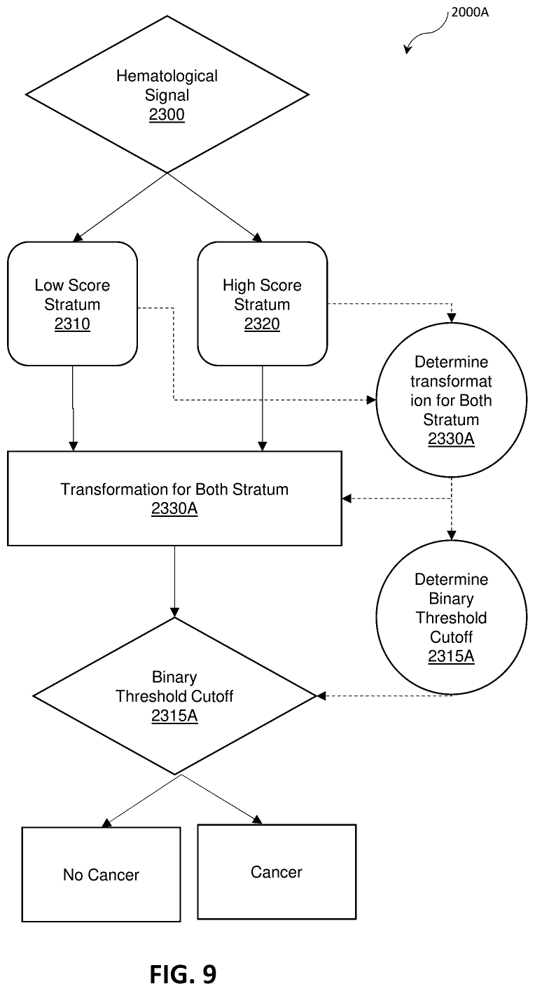

FIG. 9 illustrates an embodiment of such process 2000A, where a transformation is applied to all samples, regardless of the strata the sample belongs to. The process 2000A is similar with the process 2000, except for the following. In the process 2000A, the transformation 2330A is determined from samples from both the low prediction score stratum 2310 and the high prediction score stratum 2320. In some embodiments, the transformation is determined, such that when the transformation is applied to the samples, a fraction of the false positive high prediction score stratum samples to the total false positive samples attaches a target fraction rate. In some embodiments, such transformation is a log odds shift.

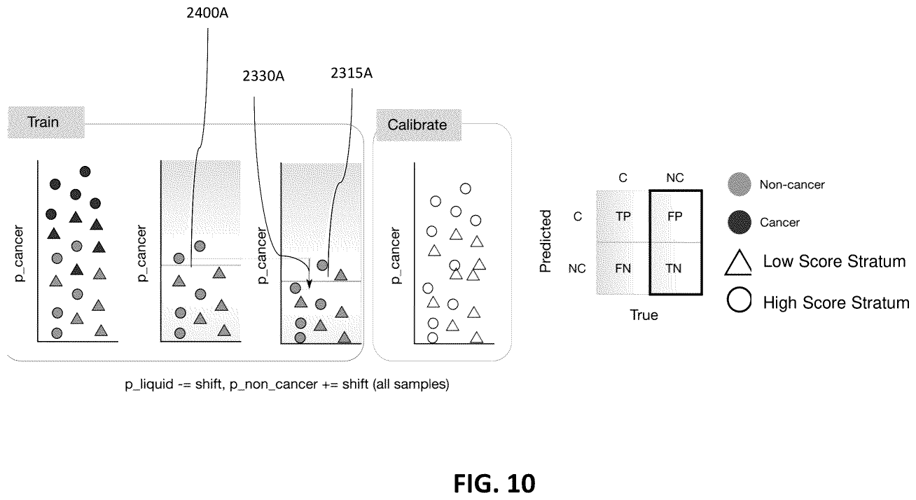

FIG. 10 illustrates an example of such transformation. Before transformation, there are two false positive high prediction score stratum (circle) samples above the initial threshold cutoff 2400A, while there is no false positive low prediction score stratum (triangle) samples. So the fraction of the false positive high prediction score stratum samples to the total false positive samples is 1 (100%). However, here, the target fraction is 0.5 (50%). After the transformation 2330A, above the final threshold cutoff 2315A, there is a one false positive high prediction score stratum sample and one false positive low prediction score stratum sample, so the fraction of the false positive high prediction score stratum sample, therefore the target fraction of 0.5 (50%) is achieved.

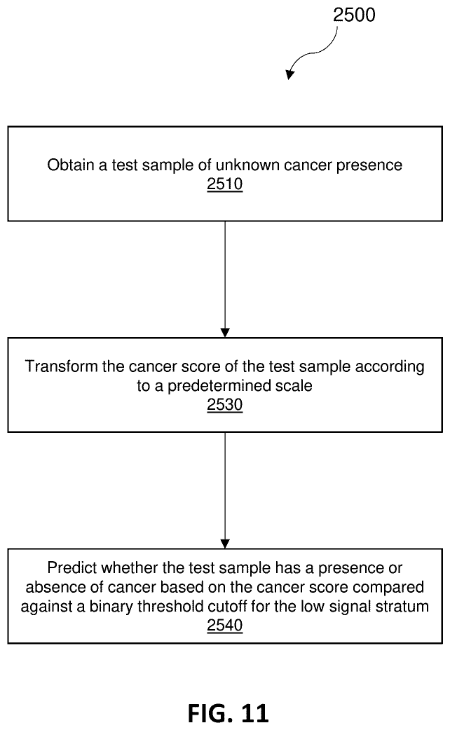

Once the analytics system determines the scale and/or formula for the transformation, and the binary threshold cutoffs, the cancer classifier may test a sample. FIG. 11 illustrates a process 2500 for the binary cancer prediction of the test sample. In step 2510, a test sample of unknown cancer presence may be obtained. In step 2530, then the cancer score of the test sample is transformed according to the transformation 2330A to provide a transformed cancer score. Then, in step 2540, the analytics system applies the binary threshold cutoff 2315A to the transformed cancer score of the test sample. It the transformed cancer score is greater than or equal to the binary threshold cutoff 2315A, then the analytics system returns a prediction of cancer presence in the test sample, and returns a prediction of no cancer otherwise.

### Deployment of Cancer Classifier

During use of the cancer classifier, the analytics system obtains a test sample from a subject of unknown cancer type. The analytics system may process the test sample comprised of DNA molecules to achieve a set of anomalous fragments. The analytics system determines a test feature vector for use by the cancer classifier according to similar principles discussed in the process 300. The analytics system calculates an anomaly score for each CpG site in a plurality of CpG sites in use by the cancer classifier. For example, the cancer classifier receives as input feature vectors inclusive of anomaly scores for 1,000 selected CpG sites. The analytics system thus determines a test feature vector inclusive of anomaly scores for the 1,000 selected CpG sites based on the set of anomalous fragments. The analytics system calculates the anomaly scores in a same manner as the training samples. In one embodiment, the analytics system defines the anomaly score as a binary score based on whether there is a hypermethylated or hypomethylated fragment in the set of anomalous fragments that encompasses the CpG site.

The analytics system then inputs the test feature vector into the cancer classifier. The function of the cancer classifier then generates a cancer prediction based on the classification parameters trained in the process 300 and the test feature vector. In the first manner, the cancer prediction is binary and selected from a group consisting of “cancer” or non-cancer;” in the second manner, the cancer prediction is selected from a group of many cancer types and “non-cancer.” In additional embodiments, the cancer prediction has predictions values for each of the many cancer types.

In some embodiments, the analytics system chains a cancer classifier trained in step 360 of the process 300 with another cancer classifier trained in step 370 or the process 300. The analytics system inputs the test feature vector into the cancer classifier trained as a binary classifier in step 360 of the process 300. The analytics system receives an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier receives the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system determines a cancer score for a test sample based on the test sample's sequencing data (e.g., methylation sequencing data, SNP sequencing data, other DNA sequencing data, RNA sequencing data, etc.). The analytics system compares the cancer score for the test sample against a binary threshold cutoff for predicting whether the test sample likely has cancer. The binary threshold cutoff can be tuned using CSO thresholding based on one or more CSO subtype classes. The analytics system may further generate a feature vector for the test sample for use in the multiclass cancer classifier to determine a cancer prediction indicating one or more likely cancer types.

It will be recognized that some or all of the figures are schematic representations for purpose of illustration.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for predicting a presence or absence of cancer in a test sample, the method comprising:
accessing the test sample having a cancer score and a prediction score for a first tissue label;
selecting one of a plurality of strata based on the prediction score, the plurality of strata including a high prediction score stratum and a low prediction score stratum;
predicting whether the test sample is associated with a presence or absence of cancer by:
transforming the cancer score of the test sample based on a predetermined transformation scale for corresponding prediction score stratum to provide a transformed cancer score; and
comparing the transformed cancer score against a predetermined binary threshold cutoff for each stratum.

2. The method of claim 1, wherein the predetermined transformation scale for low prediction score stratum is the identity transformation, and the predetermined binary threshold cutoff is identical between the low prediction score stratum and the high prediction score stratum.

3. The method of claim 2, wherein the predetermined binary threshold cutoff is determined by:
obtaining a holdout set of samples, each sample having the cancer score and the prediction score for the first tissue label;
stratifying the holdout set into the high prediction score stratum and the low prediction score stratum based on the prediction scores for the first tissue label of the holdout set of samples;
sweeping through a domain of cancer scores at a plurality of candidate binary threshold cutoffs by calculating a true positive rate and a false positive rate for each candidate binary threshold cutoff based on the cancer scores of the samples in the low prediction score stratum, and
selecting the binary threshold cutoff from the plurality of candidate binary threshold cutoffs based on a false positive budget for the low prediction score stratum and the calculated false positive rates.

4. The method of claim 3, wherein the predetermined transformation scale is determined by:
sweeping through a domain of cancer scores at a plurality of candidate binary threshold cutoffs by calculating a true positive rate and a false positive rate for each candidate binary threshold cutoff based on the cancer scores of the samples in the high prediction score stratum;
selecting the binary threshold cutoff from the plurality of candidate binary threshold cutoffs based on a false positive budget for the high prediction score stratum and the calculated false positive rates, to provide a binary threshold cutoff for the high prediction score stratum;
providing one or more candidate transformation scales that transform the binary threshold cutoff for the high prediction score stratum into the predetermined binary threshold cutoff; and
selecting the transformation scale based on a false positive budget for the high prediction score stratum.

5. The method of claim 4, wherein a false positive rate for the predetermined binary threshold cutoff based on the transformed cancer scores of the samples in the high prediction score stratum transformed according to the predetermined transformation scale, equals the false positive rate for the binary threshold cutoff for the high prediction score stratum based on the cancer scores of the samples in the high prediction score stratum before the transformation.

6. The method of claim 1, wherein the predetermined transformation scale is a monotonic transformation.

7. The method of claim 1, wherein the predetermined transformation scale is in the order of log-odds of the cancer scores.

8. The method of claim 1, wherein the test sample comprises a test feature vector determined according to methylation sequencing data of the test sample.

9. The method of claim 1, wherein the cancer score is determined by applying a binary cancer classifier to the test feature vector.

10. The method of claim 1, wherein the prediction score is a cancer signal origin (CSO) prediction determined by applying a multiclass cancer classifier to the test feature vector.

11. The method of claim 10, wherein the CSO prediction comprises a prediction value for each of a plurality of tissue labels, each prediction value indicating a likelihood that the test sample corresponds to a cancer type associated with the tissue label.

12. The method of claim 11, wherein selecting one of a plurality of strata based on the prediction score for the first tissue label comprises:
determining whether the prediction score for the first tissue label is at or above a prediction value threshold;
responsive to determining that the prediction score for the first tissue label is at or above the prediction value threshold, selecting the high prediction score stratum; and
responsive to determining that the prediction score for the first tissue label is below the prediction value threshold, selecting the low prediction score stratum.

13. The method of claim 12, wherein the CSO prediction indicates one or more top predictions of one or more tissue labels of the plurality of tissue labels, wherein a top prediction of a tissue label indicates that the test sample is predicted to have a cancer type associated with the tissue label of the top prediction.

14. The method of claim 13, wherein selecting one of the plurality of strata comprises:
determining whether the first tissue label is a top prediction;
responsive to determining that the first tissue label is the top prediction, selecting the high prediction score stratum; and
responsive to determining that the first tissue label is not the top prediction, selecting the low prediction score stratum.

15. The method of claim 14, wherein selecting one of a plurality of strata comprises:
determining whether the first tissue label is a second top prediction;
responsive to determining that the first tissue label is the second top prediction, selecting the high prediction score stratum; and
responsive to determining that the first tissue label is not the second top prediction, selecting the low prediction score stratum.

16. The method of claim 1, wherein the test sample has a prediction score for a second tissue class, wherein selecting one of a plurality of strata is further based on the prediction score for the second tissue label.

17. The method of claim 1, wherein the first tissue label is hematological cancer.

18. A method for predicting a presence or absence of cancer in a test sample, the method comprising:
accessing the test sample having a cancer score and a prediction score for a first tissue label;
selecting one of a plurality of strata based on the prediction score for the first tissue label, the plurality of strata including a first stratum for the first tissue label and a second stratum of for the first tissue label;
predicting whether the test sample is associated with a presence or absence of cancer by:
i) if the first stratum is selected for the test sample, comparing the cancer score against a predetermined binary threshold cutoff; or
ii) if the second stratum is selected for the test sample,
transforming the cancer score of the test sample based on a predetermined transformation scale to provide a transformed cancer score; and
comparing the transformed cancer score against a predetermined binary threshold cutoff,
wherein the predetermined binary threshold cutoff and the predetermined transformation scale is determined based on a holdout set of samples, each sample having a cancer/non-cancer label, the cancer score, and the prediction score for the first tissue label.

19. The method of claim 18, wherein the predetermined binary threshold cutoff is determined by:
obtaining the holdout set of samples, each sample having the cancer score and the prediction score for the first tissue label;
stratifying the holdout set into the first stratum and the second stratum based on the prediction score for the first tissue label of the holdout set of samples;
sweeping through a domain of cancer scores at a plurality of candidate binary threshold cutoffs by calculating a true positive rate and a false positive rate for each candidate binary threshold cutoff based on the cancer scores of the samples in the first stratum, and
selecting the binary threshold cutoff from the plurality of candidate binary threshold cutoffs based on a false positive budget for the first stratum and the calculated false positive rates.

20. The method of claim 19, wherein the predetermined transformation scale is determined by:
sweeping through a domain of cancer scores at a plurality of candidate binary threshold cutoffs by calculating a true positive rate and a false positive rate for each candidate binary threshold cutoff based on the cancer scores of the samples in the second stratum;
selecting the binary threshold cutoff from the plurality of candidate binary threshold cutoffs based on a false positive budget for the first stratum and the calculated false positive rates, to provide a binary threshold cutoff for the second stratum;
providing one or more candidate transformation scales that transform the binary threshold cutoff for the second stratum into the predetermined binary threshold cutoff; and
selecting the transformation scale based on a false positive budget for the second stratum.

21. The method of claim 20, wherein a false positive rate for the predetermined binary threshold cutoff based on the transformed cancer scores of the samples in the second stratum transformed according to the predetermined transformations scale, equals the false positive rate for the binary threshold cutoff for the second stratum based on the cancer scores of the samples in the second stratum before the transformation.

22. The method of claim 18, wherein the predetermined transformation scale is a monotonic transformation.

23. The method of claim 18, wherein the predetermined transformation scale is in the order of log-odds of the cancer scores.

24. The method of claim 18, wherein the test sample comprises a test feature vector determined according to methylation sequencing data of the test sample.

25. The method of claim 18, wherein the cancer score is determined by applying a binary cancer classifier to the test feature vector.

26. The method of claim 18, wherein the prediction score is a cancer signal origin (CSO) prediction determined by applying a multiclass cancer classifier to the test feature vector.

27. The method of claim 26, wherein the CSO prediction comprises a prediction value for each of a plurality of tissue labels, each prediction value indicating a likelihood that the test sample corresponds to a cancer type associated with the tissue label.

28. The method of claim 27, wherein selecting one of a plurality of strata based on the prediction score for the first tissue label comprises:
determining whether the prediction score for the first tissue label is at or above a prediction value threshold;
responsive to determining that the prediction score for the first tissue label is at or above the prediction value threshold, selecting the first stratum; and
responsive to determining that the prediction score for the first tissue label is below the prediction value threshold, selecting the second stratum.

29. The method of claim 28, wherein the CSO prediction indicates one or more top predictions of one or more tissue labels of the plurality of tissue labels, wherein a top prediction of a tissue label indicates that the test sample is predicted to have a cancer type associated with the tissue label of the top prediction.

30. The method of claim 29, wherein selecting one of the plurality of strata comprises:
determining whether the first tissue label is a top prediction;
responsive to determining that the first tissue label is the top prediction, selecting the first stratum; and
responsive to determining that the first tissue label is not the top prediction, selecting the second stratum.

31. The method of claim 30, wherein selecting one of a plurality of strata comprises:
determining whether the first tissue label is a second top prediction;
responsive to determining that the first tissue label is the second top prediction, selecting the first stratum; and
responsive to determining that the first tissue label is not the second top prediction, selecting the second stratum.

32. The method of claim 18, wherein the test sample has a prediction score for a second tissue class, wherein selecting one of a plurality of strata is further based on the prediction score for the second tissue label.

33. A method for predicting a presence or absence of cancer in a test sample, the method comprising:
accessing the test sample having a cancer score and a prediction score for a first tissue label;
transforming the cancer score of the test sample based on a predetermined transformation scale to provide a transformed cancer score; and
predicting whether the test sample is associated with a presence or absence of cancer by comparing the cancer score against a predetermined binary threshold cutoff.

34. The method of claim 33, wherein the predetermined transformation scale is determined by:
obtaining a holdout set of non-cancer samples, each sample having the cancer score and the prediction score for the first tissue label;
stratifying the holdout set into a high score stratum and a low score stratum based on the prediction scores for the first tissue label of the holdout set of non-cancer samples;
sweeping through a domain of cancer scores at a plurality of candidate transformations and a plurality of candidate binary threshold cutoffs by calculating a fraction of false positive samples from the high prediction score stratum to the total false positive samples, wherein the false positive samples have a cancer score higher than each of the binary threshold cutoffs; and
selecting the transformation and the binary threshold cutoff from the plurality of candidate binary threshold cutoffs and the plurality of candidate binary threshold cutoffs, based on a target fraction of false positive samples from the high prediction score stratum to the total false positive samples.

35. The method of claim 33, wherein the predetermined transformation scale is in the order of log-odds of the cancer scores.

36. The method of claim 33, wherein the test sample comprises a test feature vector determined according to methylation sequencing data of the test sample.

37. The method of claim 33, wherein the cancer score is determined by applying a binary cancer classifier to the test feature vector.

38. The method of claim 33, wherein the prediction score is a cancer signal origin (CSO) prediction determined by applying a multiclass cancer classifier to the test feature vector.

39. The method of claim 38, wherein the CSO prediction comprises a prediction value for each of a plurality of tissue labels, each prediction value indicating a likelihood that the test sample corresponds to a cancer type associated with the tissue label.

40. The method of claim 39, wherein selecting one of a plurality of strata based on the prediction score for the first tissue label comprises:
determining whether the prediction score for the first tissue label is at or above a prediction value threshold;
responsive to determining that the prediction score for the first tissue label is at or above the prediction value threshold, selecting the high prediction score stratum; and
responsive to determining that the prediction score for the first tissue label is below the prediction value threshold, selecting the low prediction score stratum.

41. The method of claim 40, wherein the CSO prediction indicates one or more top predictions of one or more tissue labels of the plurality of tissue labels, wherein a top prediction of a tissue label indicates that the test sample is predicted to have a cancer type associated with the tissue label of the top prediction.

42. The method of claim 41, wherein selecting one of the plurality of strata comprises:
determining whether the first tissue label is a top prediction;
responsive to determining that the first tissue label is the top prediction, selecting the high prediction score stratum; and
responsive to determining that the first tissue label is not the top prediction, selecting the low prediction score stratum.

43. The method of claim 42, wherein selecting one of a plurality of strata comprises:
determining whether the first tissue label is a second top prediction;
responsive to determining that the first tissue label is the second top prediction, selecting the high prediction score stratum; and
responsive to determining that the first tissue label is not the second top prediction, selecting the low prediction score stratum.

44. The method of claim 43, wherein the test sample has a prediction score for a second tissue class, wherein selecting one of a plurality of strata is further based on the prediction score for the second tissue label.

45. The method of claim 18, wherein the first tissue label is hematological cancer.

46. A system comprising a hardware processor and a non-transitory computer-readable storage medium storing executable instructions that, when executed by the hardware processor, cause the processor to perform steps comprising the method of claim 1.
